(19) United States
(12) Patent Application Publication
(10) Pub. No.: US 2024/0123212 A1
(43) Pub. Date: Apr. 18, 2024

(54) SPINNING COMPONENT

(71) Applicant: CareFusion 303, Inc., San Diego, CA (US)

(72) Inventors: Brett FRANKS, San Diego, CA (US); Ismael Sanchez, San Diego, CA (US)

(21) Appl. No.: 18/480,642

(22) Filed: Oct. 4, 2023

Related U.S. Application Data
(60) Provisional application No. 63/416,417, filed on Oct. 14, 2022.

(51) Int. Cl. A61M 39/10; A61M 39/22

(52) U.S. Cl. CPC A61M 39/1055; A61M 39/22; A61M 2039/1088

(57) ABSTRACT

A system for reducing twisting of medical tubing includes a spinning component and a tubing connector. The spinning component includes a first fluid channel and a protrusion, and the tubing connector includes a second fluid channel, male mating portion, and a sheath. Accordingly, the spinning component is configured to couple to the tubing connector such that the first fluid channel and the second fluid channel together form a unified fluid channel, the first fluid channel encompasses the male mating portion, the protrusion is positioned within a recess of the sheath, and the spinning component is rotatable relative to and independently of the tubing connector.

### CROSS-REFERENCE TO RELATED APPLICATION

This application claims priority to U.S. Provisional Application No. 63/416,417 (filed Oct. 14, 2022), the disclosure of which is incorporated by reference herein in its entirety.

### TECHNICAL FIELD

The present disclosure relates, generally, to connectors for medical tubing, and, more particularly, to a system that includes a spinning component configured to prevent undesired twisting of medical tubing.

### BACKGROUND

Medical treatments often include use of medical tubing. For example, many infusion treatments require flexible medical tubing to deliver a medical fluid (e.g., a saline solution, a liquid medication) from a fluid source to a patient via an intravenous (IV) catheter. Sometimes, the tubing twists and kinks—slowing or stopping the medical fluid from flowing through the tubing and to the patient. Accordingly, a system that prevents twisting and kinking of medical tubing would improve the reliability of medical treatments that involve medical tubing, such as infusion treatments.

The description provided in this section should not be assumed to be prior art merely because it is mentioned in or associated with this section. This section may include information that describes one or more aspects of the subject technology.

### SUMMARY

According to various aspects of the present disclosure, a system for reducing twisting of medical tubing may include a spinning component and a tubing connector. The spinning component may include a first end portion, a second end portion, an inner surface, and an outer surface. The first end portion may include a protrusion that extends from the outer surface in a direction away from the inner surface. The inner surface may form a fluid channel that extends through the first and second end portions. The tubing connector may include a proximal end portion, a distal end portion, and an inner surface. The inner surface may form a fluid channel that extends through the proximal and distal end portions. The distal end portion may form a male mating portion and a sheath. The sheath may include an inner surface that is spaced apart from an outer surface of the male mating portion to form a cavity therebetween. The sheath may also include a recess that extends into the inner surface of the sheath. The distal end portion of the tubing connector may be configured to couple with the first end portion of the spinning component such that the male mating portion of the tubing connector is positioned within the fluid channel of the spinning component and the protrusion of the spinning component is positioned within the recess of the sheath. The spinning component may be rotatable relative to the tubing connector.

According to various aspects of the present disclosure, a spinning component for reducing twisting of medical tubing may include a first end portion, a second end portion, an inner surface, and an outer surface. The first end portion may include a protrusion that extends from the outer surface in a direction away from the inner surface. The inner surface forms a fluid channel that extends through the first and second end portions. The spinning component may be configured to couple with a tubing connector that includes a proximal end portion, a distal end portion, and an inner surface. The inner surface may form a fluid channel that extends through the proximal and distal end portions. The distal end portion may form a male mating portion and a sheath. The sheath may include an inner surface that is spaced apart from an outer surface of the male mating portion to form a cavity therebetween. The sheath may also include a recess that extends into the inner surface of the sheath. The distal end portion of the tubing connector may be configured to couple with the first end portion of the spinning component such that the male mating portion of the tubing connector is positioned within the fluid channel of the spinning component and the protrusion of the spinning component is positioned within the recess of the sheath. The spinning component may be rotatable relative to the tubing connector.

It is to be understood that both the foregoing general description and the following detailed description are exemplary and explanatory and are intended to provide further explanation of the subject technology as claimed. It is also to be understood that other aspects may be utilized, and changes may be made without departing from the scope of the subject technology.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a perspective view of a spinning component coupled to a tubing connector and an infusion fluid bag, in accordance with some embodiments of the present disclosure.

FIG. 2 is a perspective view of a spinning component coupled to a tubing connector, in accordance with some embodiments of the present disclosure.

FIG. 3 is a cross-sectional view of the spinning component and tubing connector of FIG. 2, in accordance with some embodiments of the present disclosure.

FIG. 4 is a detail view of FIG. 3, in accordance with some embodiments of the present disclosure.

FIG. 5 is a detail view of FIG. 4, in accordance with some embodiments of the present

disclosure.

FIG. 6 is a perspective view of a spinning component, in accordance with some embodiments of the present disclosure.

FIG. 7 is a simplified cross-sectional view illustrating the spinning component and tubing connector of FIG. 2, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Various embodiments of the present disclosure are generally directed to a spinning component. The spinning component may be attached to medical tubing, a needleless connector (e.g., a Becton Dickinson SmartSite™), or a fluid source (e.g., an intravenous fluid bag). In some embodiments, the spinning component allows things attached to it to rotate independently of each other. For example, a spinning component attached to a fluid source on one end and a needleless connector on the other end would allow the needleless connector to rotate independently of the fluid source. This prevents twisting and kinking of tubing attached to one end of a spinning component due to twisting on whatever is attached to the other end of the spinning component (e.g., an operator may need to twist a needless connector in order to initiate a medical treatment, or an operator may need to twist a spike in order to attach medical tubing to a fluid bag).

While the following description is directed to use of the spinning component with medical tubing, infusion bags, and the like, it is to be understood that this description is only an example of usage and does not limit the scope of the claims.

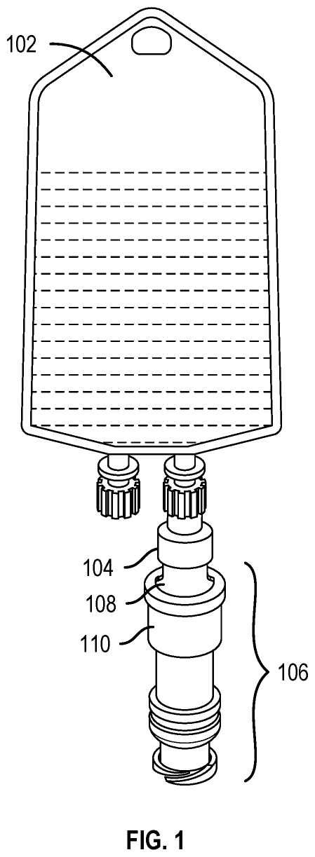

FIG. 1 is a perspective view of a spinning component 108 coupled to a tubing connector 110 and an infusion fluid bag 102, in accordance with some embodiments of the present disclosure. As denoted by the bracket in FIG. 1, in some embodiments, the spinning component 108 and the tubing connector 110 form a system 106. In some embodiments, the system 106 is configured such that the spinning component 108 and the tubing connector 110 can rotate independently of each other. In some embodiments, this functionality reduces the risk of twisting or kinking for tubing connected to the spinning component 108 and/or the tubing connector 110. In some embodiments, this functionality allows for an operator to rotate something connected to the spinning component 108 without also rotating the tubing connector 110, and vice versa.

As depicted in FIG. 1, the spinning component 108 is connected to an infusion fluid bag 102 via a nut 104. In some embodiments, the nut 104 is annular. In some embodiments, the nut 104 is cylindrical. In other embodiments, the nut 104 is prismatic (e.g., hexagonal, octagonal, etc.). In some embodiments, the nut 104 is configured to rotate relative to the spinning component such that the spinning component 108 can rotate independently of whatever is connected to the other end of the nut 104. For example, as depicted, the infusion fluid bag 102 is connected to the other end of the nut 104. Accordingly, in some embodiments, the infusion fluid bag 102 can rotate independently of the spinning component 108.

Alternatively, in some embodiments, the spinning component 108 is connected to the infusion fluid bag 102 via a coupling feature or structure, such as a snap fitting, a collar, or a compression ring. In some of these embodiments, the coupling feature or structure is configured such that the spinning component 108 can rotate independently of the infusion fluid bag 102.

In some embodiments, the spinning component 108 is connected to the infusion fluid bag 102 without the nut 104. In some embodiments, the spinning component 108 is connected to the infusion fluid bag 102 via tubing. In other embodiments, the spinning component 108 is directly connected to the infusion fluid bag 102. For example, in some embodiments, the spinning component 108 is glued to the infusion fluid bag 102. As another example, in some embodiments, the spinning component 108 includes a connector (e.g., a spike) which is inserted into the infusion fluid bag 102. As another example, in some embodiments, the spinning component 108 includes threads such that the spinning component 108 can be screwed into or onto the infusion fluid bag 102.

In some embodiments, the infusion fluid bag 102 contains a liquid (e.g., saline, medication, etc.) to be administered to a patient via a drip chamber or an infusion pump. For example, the tubing connector 110 may be connected to a drip chamber (e.g., via tubing). As another example, the tubing connector 110 may be connected to an infusion pump (e.g., via tubing). As yet another example, the tubing connector may be connected to another length of tubing (e.g., in an in-line application).

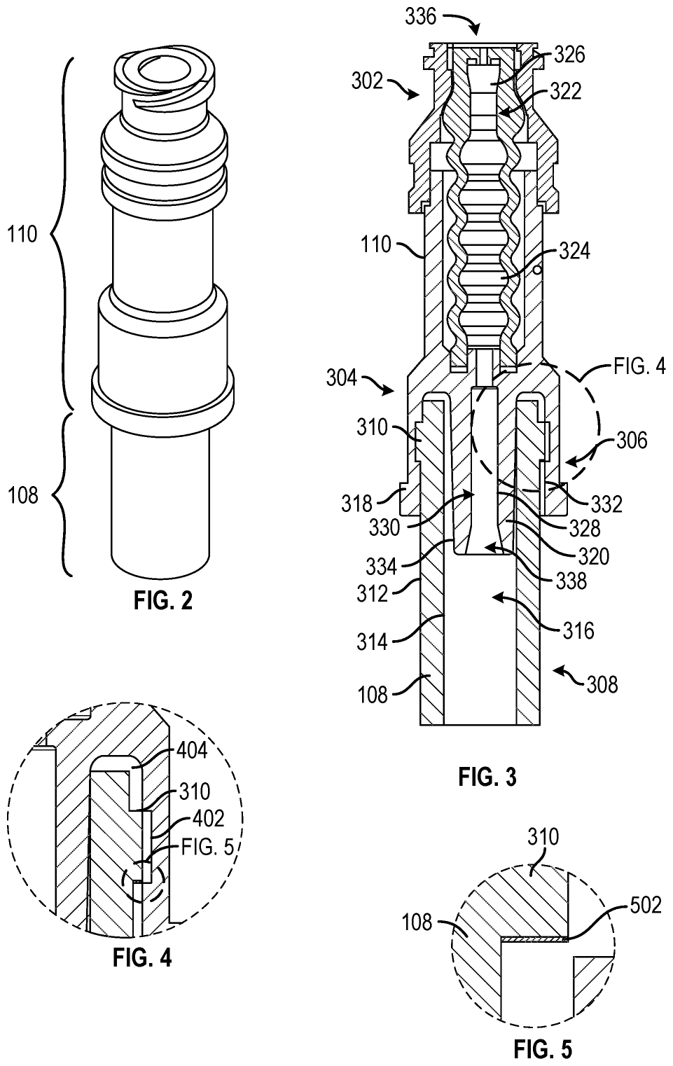

FIG. 2 is a perspective view of a spinning component 108 coupled to a tubing connector 110, in accordance with some embodiments of the present disclosure. As depicted, in some embodiments, the spinning component 108 couples with the tubing connector 110 such that a portion (e.g., distal end portion 304) of the tubing connector 110 envelopes a portion (e.g., a first end portion 306) of the spinning component 108. In some embodiments, the spinning component 108 couples with the tubing connector 110 such that a portion (e.g., a first end portion 306) of the spinning component 108 envelopes a portion (e.g., distal end portion 304) of the tubing connector. For further discussion of the coupling between the spinning component 108 and the tubing connector 110, see the paragraphs regarding FIGS. 3-5 and 7.

FIG. 3 is a cross-sectional view of the spinning component 108 and the tubing connector 110 of FIG. 2, in accordance with some embodiments of the present disclosure. As depicted, in some embodiments, the spinning component 108 couples with the tubing connector 110 such that the distal end portion 304 of the tubing connector 110 envelopes at least a portion of the first end portion 306 of the spinning component 108. In some embodiments, the spinning component 108 couples with the tubing connector 110 such that the spinning component 108 can rotate relative to the tubing connector 110.

In some embodiments, the spinning component 108 includes a first end portion 306, a second end portion 308, an inner surface 314, and an outer surface 312. In some embodiments, the first end portion includes a protrusion 310 that extends from the outer surface 312 in a direction away from the inner surface 314. In some embodiments, the inner surface 314 forms a fluid channel 316 that extends through the first 306 and second 308 end portions.

In some embodiments, the tubing connector 110 includes a proximal end portion 302, a distal end portion 304, and an inner surface 328. In some embodiments, the inner surface 328 of the tubing connector 110 forms a fluid channel 330 that extends through the proximal 302 and distal 304 end portions. In some embodiments, the distal end portion 304 forms a male mating portion 320. In some embodiments, the distal end portion 304 forms a sheath 318. In some embodiments, the sheath 318 has an inner surface 332. In some embodiments, the inner surface 332 of the sheath 318 is spaced apart from an outer surface 334 of the male mating portion 320 to form a cavity 404 therebetween.

A recess 402 extends into the inner surface 332 of the sheath 318, and is configured to receive the protrusion 310 therein when the spinning component 108 is coupled with the tubing connector 110. The recess 402 extends into the inner surface 332 of the sheath 318, in a direction that is away from the male mating portion 320. The recess 402 can be shaped as any of a channel or groove that extends into the inner surface 332 of the sheath 318 and around the circumference of the sheath 318.

In some embodiments, the sheath 318 of the tubing connector 110 is made of a flexible material. In some embodiments, this allows for the sheath 318 to expand when the tubing connector 110 and the spinning component 108 are coupled together (e.g., press-fit together). The sheath can also be formed by a discontinuous wall. In some embodiments, this permits the wall to be biased radially outward and/or radially inward. The discontinuous wall can be formed by a channel or groove that extends through inner and outer surfaces of the wall. In some embodiments, the male mating portion 320 of the tubing connector 110 is made of a flexible material. In some embodiments, this allows for the male mating portion 320 to compress when the tubing connector 110 and the spinning component 108 are coupled together (e.g., press-fit together).

In some embodiments, the outer surface of the protrusion defines a first cross-sectional width W1 (see FIG. 7), the inner surface of the sheath defines a second cross-sectional width W2 (see FIG. 7), and the inner surface of the sheath along the recess 402 defines a third cross-sectional width W3 (see FIG. 7). In some aspects of the present disclosure, the first cross-sectional width W1 is approximately equal to or greater than the second cross-sectional width W2 of the sheath. In some aspects, the first cross-sectional width W1 is between the second cross-sectional width W2 and the third cross-sectional width W3. In some embodiments, the first cross-sectional width W1 is approximately equal to the third cross-sectional width W3.

In some embodiments, the distal end portion 304 of the tubing connector 110 is configured to couple with the first end portion 306 of the spinning component 108 such that the male mating portion 320 of the tubing connector 110 is positioned within the fluid channel 316 of the spinning component 108. In some embodiments, the distal end portion 304 of the tubing connector 110 is configured to couple with the first end portion 306 of the spinning component 108 such that the protrusion 310 of the spinning component 108 fits within the recess 402 of the sheath 318. As noted above, in some embodiments, the distal end portion 304 of the tubing connector 110 is configured to couple with the first end portion 306 of the spinning component 108 such that the spinning component 108 is rotatable relative to the tubing connector 110. In some embodiments, the spinning component 108 and the tubing connector 110 couple together to form a luer lock.

In some embodiments, the fluid channel 330 formed by the inner surface 328 of the tubing connector 110 extends between a proximal port 336 at the proximal end portion 302 and a distal port 338 at the distal end portion 304. In some embodiments, the tubing connector 110 includes a collapsible valve 322 positioned within the fluid channel 330 of the tubing connector 110.

In some embodiments, the collapsible valve 322 resists a fluid flow through the proximal port 336 in a first position. For example, the collapsible valve may restrict fluid flow while in the first position, or it may restrict fluid flow altogether. In some embodiments, the collapsible valve 322 permits a fluid flow through the proximal port 336 in a second position. In some embodiments, the collapsible valve 322 includes a bellows portion 324. In some embodiments, the collapsible valve includes and a head portion 326 coupled to the bellows portion 324. In some embodiments, the head portion 326 of the collapsible valve 322 is positioned within the proximal port 336 of the tubing connector 110 when the collapsible valve 322 is in the first position. In some embodiments, the bellows portion 324 is compressed towards the distal port 338 of the tubing connector 110 when the collapsible valve 322 is in the second position.

In addition to the aforenoted embodiments, the present disclosure also contemplates embodiments in which the sheath 318 is coupled to and/or incorporated into another device, such as a drip chamber, an infusion pump, an IV set, a catheter, or another structure or device configured to direct a fluid therethrough.

FIG. 4 is a detailed view of FIG. 3, in accordance with some embodiments of the present disclosure. As depicted, in some embodiments, the distal end portion 304 of the tubing connector 110 is configured to couple with the first end portion 306 of the spinning component 108 such that the protrusion 310 of the spinning component 108 fits within and is longitudinally aligned with the recess 402 of the sheath 318.

As depicted, in some embodiments, the protrusion 310 extends uniformly from the outer surface 312 of the spinning component 108 in a direction away from the inner surface 314. However, in some embodiments, the protrusion does not extend uniformly away from the outer surface 312 in a direction away from the inner surface 314. For example, in some embodiments, the protrusion 310 tapers such that a portion of the protrusion 310 nearest to the second end portion 308 extends further from the outer surface 312 than does a portion of the protrusion 310 furthest from the second end portion 308. In other words, in some embodiments, the protrusion 310 tapers away from the second end portion 308 of the spinning component 108 such that a cross-sectional width of the protrusion decreases in a direction from the second end portion 308 toward the first end portion 306. As another example, in some embodiments, the protrusion 310 is rounded such that portions of the protrusion 310 nearest to and furthest from the second end portion 308 do not extend as far as another portion (e.g., a middle portion) of the protrusion 310. In some embodiments, the recess 402 of the tubing connector is sized to compliment the protrusion 310 of the spinning component 108.

FIG. 5 is a detailed view of FIG. 4, in accordance with some embodiments of the present disclosure. As depicted, in some embodiments, the spinning component includes a ring 502 (e.g., an O-ring) coupled to the outer surface 312 of the spinning component 108. In some embodiments, the ring 502 abuts a side of the protrusion 310 nearest the second end portion 308 of the spinning component 108. In some embodiments, the ring 502 abuts a side of the protrusion 310 furthest from the second end portion 308 of the spinning component 108. In some embodiments, the spinning component 108 includes multiple rings. For example, in some embodiments, the spinning component 108 includes a ring 502 on the side of the protrusion 310 nearest the second end portion 308 of the spinning component 108 and another ring 502 on the side of the protrusion 310 furthest from the second end portion 308 of the spinning component 108.

In some embodiments, the ring 502 abuts the recess of the tubing connector when the spinning component 108 is attached to the tubing connector. In some embodiments, the ring 502 comprises a lubricant (e.g., a lubricated O-ring). In some embodiments, the ring 502 serves to decrease friction between the spinning component 108 and the tubing connector 110.

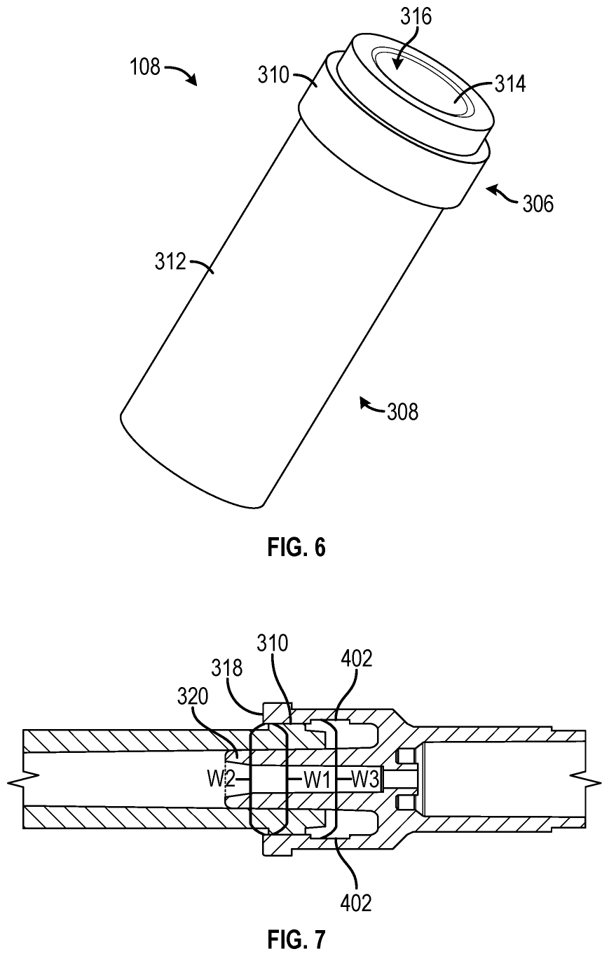

FIG. 6 is a perspective view of a spinning component 108, in accordance with some embodiments of the present disclosure. As depicted, in some embodiments, the spinning component 108 includes a first end portion 306, a second end portion 308, an inner surface 314, and an outer surface 312. In some embodiments, the first end portion includes a protrusion 310 that extends from the outer surface 312 in a direction away from the inner surface 314. In some embodiments, the inner surface 314 forms a fluid channel 316 that extends through the first 306 and second 308 end portions.

As depicted, in some embodiments, the shape of the spinning component 108 is cylindrical. However, in some embodiments, the shape of the spinning component is prismatic (e.g., an octagonal prism, a hexagonal prism, a rectangular prism, or a triangular prism). Further, though FIG. 6 depicts the first 306 and second 308 end portions as being roughly the same shape, in some embodiments, the first 306 and second 308 end portions are different shapes (e.g., cylindrical or prismatic). For further discussion of the shape of the spinning component 108, specifically with respect to the protrusion 310, see the paragraphs regarding FIG. 4.

FIG. 7 is a simplified cross-sectional view of the spinning component 108 and tubing connector 110 of FIG. 2, in accordance with some embodiments of the present disclosure. As depicted, the spinning component 108 and the tubing connector 110 are configured to couple together. In some embodiments, the spinning component 108 and the tubing connector 110 are coupled together by moving the first end portion 306 of the spinning component 108 and the distal end portion 304 of the tubing connector 110 toward each other.

As the first end portion 306 and the distal end portion 304 move toward each other, the male mating portion 320 of the tubing connector 110 enters into the fluid channel 316 of the spinning component 108 (e.g., forming a luer lock). Additionally, the protrusion 310 of the spinning component 108 enters into the cavity 404 formed by the sheath 318 and the male mating portion 320 of the tubing connector 110. In so doing, the protrusion 310 contacts the sheath 318 and the male mating portion 320, causing the sheath 318 to expand, or the male mating portion 320 to compress, to allow for the protrusion 310 to slide towards and into the recess 402 of the tubing connector 110.

After the protrusion 310 enters the recess 402, the sheath 318 may return to its original, unexpanded position Likewise, the male mating portion 320 may return to its original, uncompressed position. However, in some embodiments, the male mating portion 320 or the sheath 318 may remain in a flexed (e.g., expanded, compressed) position to ensure a tight seal between the spinning component 108 and the tubing connector 110.

Various examples of aspects of the disclosure are described as numbered clauses (1, 2, 3, etc.) for convenience. These are provided as examples and do not limit the subject technology. Identification of the figures and reference numbers are provided below merely as examples for illustrative purposes, and the clauses are not limited by those identifications.
Clause 1: A system for reducing twisting of medical tubing, comprising: a spinning component comprising a first end portion, a second end portion, an inner surface, and an outer surface, wherein the first end portion includes a protrusion that extends from the outer surface in a direction away from the inner surface, and the inner surface forms a fluid channel that extends through the first and second end portions; and a tubing connector comprising a proximal end portion, a distal end portion, and an inner surface, wherein the inner surface forms a fluid channel that extends through the proximal and distal end portions, and the distal end portion forms a male mating portion and a sheath, the sheath having an inner surface that is spaced apart from an outer surface of the male mating portion to form a cavity therebetween, and a recess that extends into the inner surface of the sheath; wherein, the distal end portion of the tubing connector is configured to couple with the first end portion of the spinning component such that the male mating portion of the tubing connector is positioned within the fluid channel of the spinning component and the protrusion of the spinning component is positioned within the recess of the sheath, and the spinning component is rotatable relative to the tubing connector.
Clause 2: The system of clause 1, wherein the second end portion of the spinning component is configured to couple with an infusion fluid bag.
Clause 3: The system of any of clauses 1-2, wherein the second end portion comprises a nut that can rotate independently of the spinning component.
Clause 4: The system of any of clauses 1-3, wherein the spinning component comprises a ring coupled to the outer surface of the spinning component.
Clause 5: The system of clause 4, wherein the ring abuts a first side of the protrusion of the first end portion of the spinning component.
Clause 6: The system of any of clauses 4-5, wherein the ring abuts the recess of the tubing connector when the spinning component is attached to the tubing connector.
Clause 7: The system of any of clauses 4-6, wherein the ring comprises a lubricant.
Clause 8: The system of clauses 5-7, wherein the spinning component further comprises another ring coupled to the outer surface of the spinning component, wherein the other ring abuts a second side of the protrusion, different from the first side of the protrusion.
Clause 9: The system of clause 8, wherein the other ring abuts the recess of the tubing connector when the spinning component is attached to the tubing connector.
Clause 10: The system of any of clauses 1-9, wherein: the fluid channel of the tubing connector further extends between a proximal port at the proximal end portion and a distal port at the distal end portion; and the tubing connector comprises a collapsible valve positioned within the fluid channel of the tubing connector to resist a fluid flow through the proximal port in a first position, and permit the fluid flow through the proximal port in a second position.
Clause 11: The system of clause 10, wherein the collapsible valve comprises a bellows portion and a head portion coupled to the bellows portion.
Clause 12: The system of clause 11, wherein the head portion of the collapsible valve is positioned within the proximal port of the tubing connector when the collapsible valve is in the first position.
Clause 13: The system of any of clauses 11-12, wherein the bellows portion is compressed towards the distal port of the tubing connector when the collapsible valve is in the second position.
Clause 14: The system of any of clauses 1-13, wherein a cross-sectional width of the male mating portion of the tubing connector tapers towards the distal end portion of the tubing connector.
Clause 15: The system of any of clauses 1-14, wherein the spinning component and the tubing connector couple together to form a luer lock.
Clause 16: A spinning component for reducing twisting of medical tubing, comprising: a first end portion, a second end portion, an inner surface, and an outer surface, wherein the first end portion includes a protrusion that extends from the outer surface in a direction away from the inner surface, and the inner surface forms a fluid channel that extends through the first and second end portions; and wherein the spinning component is configured to couple with a tubing connector comprising a proximal end portion, a distal end portion, and an inner surface, wherein the inner surface forms a fluid channel that extends through the proximal and distal end portions, and the distal end portion forms a male mating portion and a sheath, the sheath having an inner surface that is spaced apart from an outer surface of the male mating portion to form a cavity therebetween, and a recess that extends into the inner surface of the sheath; wherein, the distal end portion of the tubing connector is configured to couple with the first end portion of the spinning component such that the male mating portion of the tubing connector is positioned within the fluid channel of the spinning component and the protrusion of the spinning component is positioned within the recess of the sheath, and the spinning component is rotatable relative to the tubing connector.
Clause 17: The spinning component of clause 16, wherein the second end portion of the spinning component is configured to couple with an infusion fluid bag.
Clause 18: The spinning component of any of clauses 16-17, wherein the second end portion comprises a nut that can rotate independently of the spinning component.
Clause 19: The spinning component of any of clauses 16-18, wherein the spinning component comprises a ring coupled to the outer surface of the spinning component.
Clause 20: The spinning component of clause 19, wherein the ring abuts a first side of the protrusion of the first end portion of the spinning component.
Clause 21: The spinning component of any of clauses 19-20, wherein the ring abuts the recess of the tubing connector when the spinning component is attached to the tubing connector.
Clause 22: The spinning component of any of clauses 19-21, wherein the ring comprises a lubricant.
Clause 23: The spinning component of clauses 20-22, wherein the spinning component further comprises another ring coupled to the outer surface of the spinning component, wherein the other ring abuts a second side of the protrusion, different from the first side of the protrusion.
Clause 24: The spinning component of clause 23, wherein the other ring abuts the recess of the tubing connector when the spinning component is attached to the tubing connector.
Clause 25: The spinning component of any of clauses 16-24, wherein: the fluid channel of the tubing connector further extends between a proximal port at the proximal end portion and a distal port at the distal end portion; and the tubing connector comprises a collapsible valve positioned within the fluid channel of the tubing connector to resist a fluid flow through the proximal port in a first position, and permit the fluid flow through the proximal port in a second position.
Clause 26: The spinning component of clause 25, wherein the collapsible valve comprises a bellows portion and a head portion coupled to the bellows portion.
Clause 27: The spinning component of clause 26, wherein the head portion of the collapsible valve is positioned within the proximal port of the tubing connector when the collapsible valve is in the first position.
Clause 28: The spinning component of any of clauses 26-27, wherein the bellows portion is compressed towards the distal port of the tubing connector when the collapsible valve is in the second position.
Clause 29: The spinning component of any of clauses 16-28, wherein a cross-sectional width of the male mating portion of the tubing connector tapers towards the distal end portion of the tubing connector.
Clause 30: The spinning component of any of clauses 16-29, wherein the spinning component and the tubing connector couple together to form a luer lock.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations, or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings, and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. §§ 101, 102, or 103, nor should they be interpreted in such a way.

What is claimed is:

1. A system for reducing twisting of medical tubing, comprising:
a spinning component comprising a first end portion, a second end portion, an inner surface, and an outer surface, wherein the first end portion includes a protrusion that extends from the outer surface in a direction away from the inner surface, and the inner surface forms a fluid channel that extends through the first and second end portions; and
a tubing connector comprising a proximal end portion, a distal end portion, and an inner surface, wherein the inner surface forms a fluid channel that extends through the proximal and distal end portions, and the distal end portion forms a male mating portion and a sheath, the sheath having an inner surface that is spaced apart from an outer surface of the male mating portion to form a cavity therebetween, and a recess that extends into the inner surface of the sheath;
wherein, the distal end portion of the tubing connector is configured to couple with the first end portion of the spinning component such that the male mating portion of the tubing connector is positioned within the fluid channel of the spinning component and the protrusion of the spinning component is positioned within the recess of the sheath, and the spinning component is rotatable relative to the tubing connector.

2. The system of claim 1, wherein the second end portion of the spinning component is configured to couple with an infusion fluid bag.

3. The system of claim 1, wherein the second end portion comprises a nut that can rotate independently of the spinning component.

4. The system of claim 1, wherein the spinning component comprises a ring coupled to the outer surface of the spinning component.

5. The system of claim 4, wherein the ring abuts a first side of the protrusion of the first end portion of the spinning component.

6. The system of claim 4, wherein the ring abuts the recess of the tubing connector when the spinning component is attached to the tubing connector.

7. The system of claim 4, wherein the ring comprises a lubricant.

8. The system of claim 5, wherein the spinning component further comprises another ring coupled to the outer surface of the spinning component, wherein the other ring abuts a second side of the protrusion, different from the first side of the protrusion.

9. The system of claim 8, wherein the other ring abuts the recess of the tubing connector when the spinning component is attached to the tubing connector.

10. The system of claim 1, wherein:
the fluid channel of the tubing connector further extends between a proximal port at the proximal end portion and a distal port at the distal end portion; and
the tubing connector comprises a collapsible valve positioned within the fluid channel of the tubing connector to resist a fluid flow through the proximal port in a first position, and permit the fluid flow through the proximal port in a second position.

11. The system of claim 10, wherein the collapsible valve comprises a bellows portion and a head portion coupled to the bellows portion.

12. The system of claim 11, wherein the head portion of the collapsible valve is positioned within the proximal port of the tubing connector when the collapsible valve is in the first position.

13. The system of claim 11, wherein the bellows portion is compressed towards the distal port of the tubing connector when the collapsible valve is in the second position.

14. The system of claim 1, wherein a cross-sectional width of the male mating portion of the tubing connector tapers towards the distal end portion of the tubing connector.

15. The system of claim 1, wherein the spinning component and the tubing connector couple together to form a luer lock.

16. A spinning component for reducing twisting of medical tubing, comprising:
a first end portion, a second end portion, an inner surface, and an outer surface, wherein the first end portion includes a protrusion that extends from the outer surface in a direction away from the inner surface, and the inner surface forms a fluid channel that extends through the first and second end portions; and
wherein the spinning component is configured to couple with a tubing connector comprising a proximal end portion, a distal end portion, and an inner surface, wherein the inner surface forms a fluid channel that extends through the proximal and distal end portions, and the distal end portion forms a male mating portion and a sheath, the sheath having an inner surface that is spaced apart from an outer surface of the male mating portion to form a cavity therebetween, and a recess that extends into the inner surface of the sheath;
wherein, the distal end portion of the tubing connector is configured to couple with the first end portion of the spinning component such that the male mating portion of the tubing connector is positioned within the fluid channel of the spinning component and the protrusion of the spinning component is positioned within the recess of the sheath, and the spinning component is rotatable relative to the tubing connector.

17. The spinning component of claim 16, wherein the second end portion comprises a nut that can rotate independently of the spinning component.

18. The spinning component of claim 16, wherein the spinning component comprises a ring coupled to the outer surface of the spinning component, and the ring abuts (i) a first side of the protrusion of the first end portion of the spinning component and (ii) the recess of the tubing connector when the spinning component is attached to the tubing connector.

19. The spinning component of claim 18, wherein the spinning component further comprises another ring coupled to the outer surface of the spinning component, wherein the other ring abuts (i) a second side of the protrusion, different from the first side of the protrusion and (ii) the recess of the tubing connector when the spinning component is attached to the tubing connector.

20. The spinning component of claim 16, wherein:
the fluid channel of the tubing connector further extends between a proximal port at the proximal end portion and a distal port at the distal end portion; and
the tubing connector comprises a collapsible valve positioned within the fluid channel of the tubing connector to resist a fluid flow through the proximal port in a first position, and permit the fluid flow through the proximal port in a second position.
